(19) United States
(12) Patent Application Publication
(10) Pub. No.: US 2016/0367604 A1
(43) Pub. Date: Dec. 22, 2016

(54) USE OF EGG YOLK OIL AND THE BENZOPYRENE-ELIMINATED EGG YOLK OIL IN THE TREATMENT OF ALLERGIC RHINITIS

(71) Applicant: Harbin Medical University, Heilongjiang Province (CN)

(72) Inventor: De-Li DONG, Heilongjiang (CN)

(73) Assignee: Harbin Medical University, Heilongjiang Province (CN)

(21) Appl. No.: 15/143,850

(22) Filed: May 2, 2016

(30) Foreign Application Priority Data
Jun. 17, 2015 (CN) 201510337361.X

(51) Int. Cl. A61K 35/56; A61K 9/00

(52) U.S. Cl. CPC A61K 35/56; A61K 9/0043

(57) ABSTRACT

The application of egg yolk oil, as well as benzopyrene-eliminated egg yolk oil, is prepared for medicaments for the treatment of allergic rhinitis. By topical application of egg yolk oil in the nasal cavity, symptoms of allergic rhinitis could be improved significantly. After topical treatment with egg yolk oil, levels of inflammatory cytokines IL-4 and IgE antibodies in lymphocytes of model mice with allergic rhinitis may be reduced. The method discloses a new application of egg yolk oil for the treatment of allergic rhinitis. In order to avoid the presence of residual benzopyrene (which is potentially carcinogenic) in egg yolk oil extracted by heating yolk, the method prepares a medicament for treating allergic rhinitis with benzopyrene-eliminated egg yolk oil, thereby avoiding the potential carcinogenic risk of benzopyrene residue.

### TECHNICAL FIELD

The present invention relates to the use of egg yolk oil, more particularly, to the use of egg yolk oil and the benzopyrene-eliminated egg yolk oil in the preparation of a medicament for the treatment of allergic rhinitis. This invention belongs to the field of biomedical technology.

### BACKGROUND

Allergic rhinitis is a common disease worldwide, it manifests itself with the following symptoms: paroxysmal sneezing, watery nasal mucus, nasal congestion, nasal itching and even hyposmia, etc., it causes great suffering to the patients. Drugs currently used for treating allergic rhinitis include antihistamines, corticosteroids, anti-leukotriene drugs, nasal decongestants, etc. All these drugs have some side effects, and repeated drug-applications are needed to control the symptoms. The present inventors have found that egg yolk oil topically applied to nasal cavity had a significant effect on allergic rhinitis, could significantly improve symptoms of allergic rhinitis in mice. Egg yolk oil is derived from natural animal sources, it has no side effects. On the basis of above-mentioned, the present invention provides new medicaments to treat allergic rhinitis, that is, topically applying egg yolk oil in nasal cavity to treat allergic rhinitis.

### SUMMARY OF THE INVENTION

The present invention proposes a novel medicament and a method for the treatment of allergic rhinitis, that is, a new use of egg yolk oil as a treatment for allergic rhinitis, and a method of topically smearing egg yolk oil in nasal cavity for the treatment.

The present invention adopts the following technical solutions:

Application of yolk oil in the preparation of a medicament for the treatment of allergic rhinitis, as well as application of benzopyrene-eliminated egg yolk oil in the preparation of a medicament for the treatment of allergic rhinitis.

In the present invention, the preferable method to use egg yolk oil is to topically smear it in nasal cavity.

In the present invention, the preferable method to use benzopyrene-eliminated egg yolk oil is to topically smear it in nasal cavity.

In the present invention, the preferable method to prepare egg yolk oil mentioned above is as following: cook eggs thoroughly, pick the yolks, dry the yolks at 80˜110° C. and smash them into powder, bake the powder at 210˜260° C. to obtain the egg yolk oil.

In the present invention, the preferable method to prepare benzopyrene-eliminated egg yolk oil mentioned above is as following:

(1) cook eggs thoroughly, pick the yolks, dry the yolks at 80˜110° C. and smash them into powder, bake the powder at 210˜260° C. to obtain the egg yolk oil.

(2) Weigh out activated carbon, which is 1.5% of the weight of egg yolk oil, mix the activated carbon with the egg yolk oil which has been filtered through gauze, stir the mixture at 70° C. for 2˜3 hours, suction filter through filtration paper and filtration cloth to obtain benzopyrene-eliminated egg yolk oil.

In the present invention, the mice model with nasal allergic rhinitis is prepared by intraperitoneal injection of ovalbumin and then intranasal instillation of ovalbumin, egg yolk oil treatment is simultaneously implemented with intranasal instillation of ovalbumin. It was found that topically application of egg yolk oil had a significant effect on allergic rhinitis, could significantly improve symptoms in model mice with nasal allergic rhinitis, as well as could reduce the levels of lymphocyte inflammatory cytokines IL-4 and IgE antibody in these model mice. Therefore, on the basis of above mentioned, the present invention proposes an application of egg yolk oil in the preparation of a medicament for the treatment of nasal allergic rhinitis.

To avoid presence of residual benzopyrene (which is potentially carcinogenic) in the egg yolk oil extracted by heating yolk, the present invention further proposes the application of benzopyrene-eliminated egg yolk oil in the preparation of a medicament for treating nasal allergic rhinitis, thereby avoiding potential carcinogenic risk of residual benzopyrene.

### DESCRIPTION OF THE ATTACHED FIGURES

FIG. 1 is the histogram of the reduction of lymphocyte inflammatory cytokines IL-4 levels in model mice with nasal allergic rhinitis after treatments with egg yolk oil as well as benzopyrene-eliminated egg yolk oil, where **: P<0.01 vs blank control group; ##: P<0.01 vs nasal sensitized group (nasal allergic rhinitis model group).

FIG. 2 is the histogram of the reduction of lymphocyte IgE antibody levels in model mice with nasal allergic rhinitis after treatments with egg yolk oil and benzopyrene-eliminated egg yolk oil, where **: P<0.01 vs blank control group; #: P<0.05 vs nasal sensitized group (nasal allergic rhinitis model group).

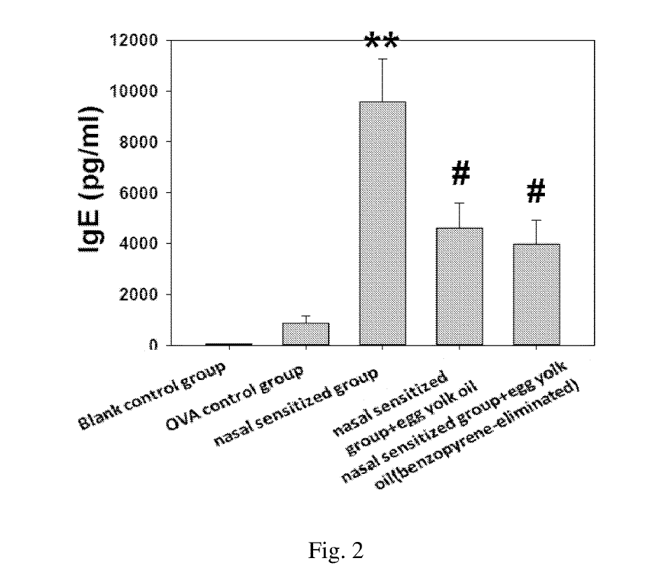

### EMBODIMENTS

In conjunction with the specific embodiments below, the present invention is further demonstrated, advantages and features of the invention will become more apparent with the demonstrations. However, these examples are only used for exemplification and are not exert any restrictions on the scope of the invention. The skilled technicians will appreciate that, without departing from the spirit and scope of the present invention, the details and procedures of technical solutions of the present invention can be modified or replaced, however, these modifications and alterations fall within the scope of protection to the present invention.

Preparations of the egg yolk oil used in the embodiments of present invention were as following: cook eggs thoroughly, pick the yolks, dry the yolks at 80˜10° C. and smash them into powder, bake the yolk powder at 210˜260° C. to obtain the egg yolk oil.

Preparations of the benzopyrene-eliminated egg yolk oil used in the embodiments of the present invention were as following:

(1) Cook eggs thoroughly, pick the yolks, dry the yolks at 80˜110° C. and smash them into powder, bake the yolk powder at 210˜260° C. to obtain the egg yolk oil.

(2) Weigh out activated carbon, which is 1.5% of the weight of egg yolk oil, mix the activated carbon with the egg yolk oil which has been filtered through gauze, stir the mixture at 70° C. for 2˜3 hours, suction filter through filtration paper and filtration cloth to obtain benzopyrene-eliminated egg yolk oil.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding Chinese Application No. 201510337361.X, filed Jun. 17, 2015 are incorporated by reference herein.

### Example 1

Egg yolk oil and benzopyrene-eliminated egg yolk oil used as medication for the treatment of allergic rhinitis, and method of application

1. Animals and Reagents:

BALB/c female mice (strain: BALB/c, purchased from Beijing Vital River Laboratory Animal Technology Co., Ltd.), ovalbumin (Sigma-aldrich)

2. Experimental Procedures:

20 mice were randomly selected, and randomly divided into five groups (blank control group, ovalbumin (OVA) group, OVA+nasal sensitized group, OVA+nasal sensitized group+egg yolk oil group, OVA+nasal sensitized group+benzopyrene-eliminated egg yolk oil group), 4 mice each. Ovalbumin (OVA) group, OVA+nasal sensitized group, OVA+nasal sensitized group+egg yolk oil group, OVA+nasal sensitized group+benzopyrene-eliminated egg yolk oil group were intraperitoneally injected with ovalbumin mixed solution (10 μg OVA, emulsification into 2 mg Al(OH)3, dissolved in 0.5 ml PBS) in the first as well as the seventh days. To each of the OVA group mice, 10 μl PBS and same volume of the solvent of the treatment medications used below were dropped into each nostril using micropipette on days 14, 15, 16, 21, 22, 23, 28, 29, and 30, respectively.

To each of the OVA+nasal ssensitized group mice, 10 μl OVA (1 mg/ml, without adding Al(OH)3 in this case) and same volume of the solvent of the treatment medications used below were dropped into each nostril using a micropipette, on the days 14, 15, 16, 21, 22, 23, 28, 29, and 30. To each of the OVA+nasal sensitized group+egg yolk oil group mice, as well as the OVA+nasal sensitized group+benzopyrene-eliminated egg yolk oil group mice, 10 μl OVA (1 mg/ml), and 5 μl treatment medications (egg yolk oil or benzopyrene-eliminated egg yolk oil) were simultaneously dropped into each nostril on the days 14, 15, 16, 21, 22, 23, 28, 29, and 30 by using micropipette.

On the day 31, i.e., 24 hours after administration, sacrifice mice. Sample and quickly freeze the nasal palates containing the nasal mucosa of the mice. Separate cervical lymph nodes of the mice and made into a single cell suspension (1×106/ml) through a 100-μm cell sieve. Culture the cell suspension in a serum-free cell cryopreserved culture medium (RPMI) containing 5% FCS, gentamycin, and β-mercaptoethanol, then use 10 μg/ml OVA to re-stimulate for 4 days. After above treatments, collect the supernatant.

Use ELISA assay to determine lymph IL-4 and IgE contents in the supernatant of lymph cell culture.

3. Results

3.1. Egg yolk oil and benzopyrene-eliminated egg yolk oil reduce the level of inflammatory cytokines IL-4 in model mice with allergic nasal rhinitis;

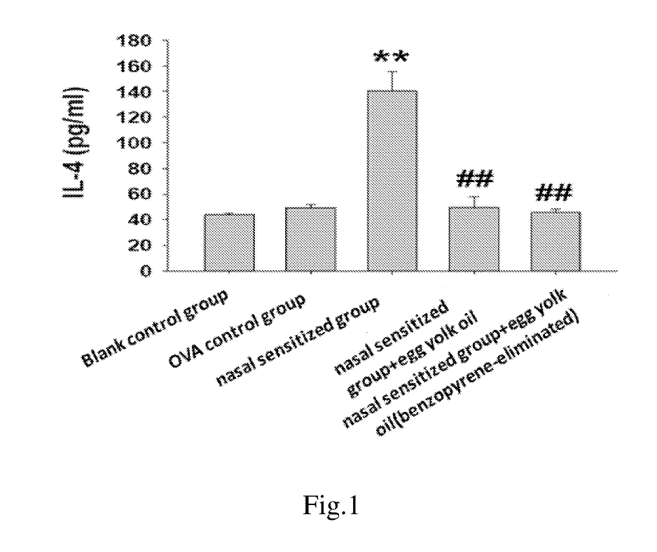

Sample the cervical lymph nodes of mice and made into single cell suspension (1×106/ml) through a 100-μm cell sieve. Culture the cell suspension in a serum-free cell cryopreserved culture medium (RPMI) containing 5% FCS, gentamycin, and β-mercaptoethanol, then use 10 μg/ml OVA to re-stimulate for 4 days. After above treatments, collect the supernatant. Use ELISA assay to determine IL-4 content in the supernatant of lymph cell culture. The results showed that topically applications of both egg yolk oil and benzopyrene-eliminated egg yolk oil into nasal cavity can reduce lymphocyte IL-4 release level in model mice with allergic nasal rhinitis. The results are shown in FIG. 1.

3.2 Egg yolk oil and benzopyrene-eliminated egg yolk oil reduce IgE’

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for the application of egg yolk oil in the preparation of a medicament for the treatment of allergic rhinitis.

2. A method for the application of benzopyrene-eliminated egg yolk oil in the preparation of a medicament for treating allergic rhinitis.

3. The method according to claim 1, wherein the application method of egg yolk oil is to topically smear it in nasal cavity.

4. The method according to claim 2, wherein the application method of benzopyrene-eliminated egg yolk oil is to topically smear it in nasal cavity.

5. The method according to claim 1, wherein the egg yolk oil is prepared as follows: cook eggs thoroughly, pick the yolks, dry the yolks at 80˜110° C. and smash them into powder, bake the powder at 210˜260° C. to obtain egg yolk oil.

6. The method according to claim 2, wherein the preparation method of benzopyrene-eliminated egg yolk oil mentioned above is as follows:
(1) cook eggs thoroughly, pick the yolks, dry the yolks at 80˜110° C. and smash them into powder, bake the powder at 210˜60° C. to obtain egg yolk oil;
(2) Weigh out activated carbon, which is 1.5% of the weight of egg yolk oil, mix the activated carbon with the egg yolk oil having been filtered through gauze, stir the mixture at 70° C. for 2˜3 hours, suction filter through filtration paper and filtration cloth to obtain benzopyrene-eliminated egg yolk oil.
